SEQUENCE LISTING (10 sequences)

SEQ ID NO: 1 - DNA, 423 bp, Homo sapiens
catatgcaaggtcaagatcgccacatgattagaatgcgtcaacttatagatattgttgatcagctgaaaaattatgtgaatgacctggttccggaattcctgccggctccggaagatgttgagaccaactgtgagtggtccgctttctcctgtttccagaaagcccagctgaaatccgcaaacaccggtaacaacgaacgtatcatcaacgtttccattaaaaaactgaaacgtaaaccgccgtccaccaacgcaggtcgtcgtcagaaacaccgtctgacctgcccgtcctgtgattcttatgagaaaaaaccgccgaaagaattcctggaacgtttcaaatccctgctgcagaaaatgattcaccagcacctgtcctctcgtacccacggttccgaagattcctgatgatttggcggatcc

SEQ ID NO: 2 - protein, 133 aa, Homo sapiens [sequence omitted]

SEQ ID NO: 3 - DNA, 418 bp, Artificial Sequence
  Synthetic
ccaaggtcaagatcgccacatgattagaatgcgtcaacttatagatattgttgatcagctgaaaaattatgtgaatgacctggttccggaattcctgccggctccggaagatgttgagaccaactgtgagtggtccgctttctcctgtttccagaaagcccagctgaaatccgcaaacaccggtaacaacgaacgtatcatcaacgtttccattaaaaaactgaaacgtaaaccgccgtccaccaacgcaggtcgtcgtcagaaacaccgtctgacctgcccgtcctgtgattcttatgagaaaaaaccgccgaaagaattcctggaacgtttcaaatccctgctgcagaaaatgattcaccagcacctgtcctctcgtacccacggttccgaagattcctgatgatttggcggatcc

SEQ ID NO: 4 - protein, 405 aa, Artificial Sequence
  Synthetic
CAAGGTCAAGATCGCCACATGATTAGAATGCGTCAACTTATAGATATTGTTGATCAGCTGAAAAATTATGTGAATGACCTGGTTCCGGAATTCCTGCCGGCTCCGGAAGATGTTGAGACCAACTGTGAGTGGTCCGCTTTCTCCTGTTTCCAGAAAGCCCAGCTGAAATCCGCAAACACCGGTAACAACGAACGTATCATCAACGTTTCCATTAAAAAACTGAAACGTAAACCGCCGTCCACCAACGCAGGTCGTCGTCAGAAACACCGTCTGACCTGCCCGTCCTGTGATTCTTATGAGAAAAAACCGCCGAAAGAATTCCTGGAACGTTTCAAATCCCTGCTGCAGAAAATGATTCACCAGCACCTGTCCTCTCGTACCCACGGTTCCGAAGATTCCTGATGA

SEQ ID NO: 5 - DNA, 13 bp, Artificial Sequence
  Synthetic
tatgaaaatgaaa

SEQ ID NO: 6 - protein, 4 aa, Artificial Sequence
  Synthetic
MKMK

SEQ ID NO: 7 - DNA, 13 bp, Artificial Sequence
  Synthetic
tatgaaaagcaaa

SEQ ID NO: 8 - protein, 4 aa, Artificial Sequence
  Synthetic
MKSK

SEQ ID NO: 9 - DNA, 13 bp, Artificial Sequence
  Synthetic
tatgaaaaccaaa

SEQ ID NO: 10 - protein, 4 aa, Artificial Sequence
  Synthetic
MKTK

(19) United States
(12) Patent Application Publication
(10) Pub. No.: US 2010/0041153 A1
(43) Pub. Date: Feb. 18, 2010

(54) Expression of Proteins in E. Coli

(75) Inventors: Helle Woldike, Lynge (DK); Christine Bruun Schiodt, Bronshoj (DK)

Correspondence Address: NOVO NORDISK, INC.;INTELLECTUAL PROPERTY DEPARTMENT, 100 COLLEGE ROAD WEST, PRINCETON, NJ, 08540, US

(73) Assignee: NOVO NORDISK HEALTHCARE AG, Zurich (CH)

(21) Appl. No.: 12/528,901

(22) PCT Filed: Feb. 22, 2008
(86) PCT No.: PCT/EP08/52193

§ 371 Date: Oct. 16, 2009

(30) Foreign Application Priority Data
Mar. 1, 2007 (EP) 07103318.7

(51) Int. Cl. C12N 15/70; C12N 1/21; C07K 14/195

(52) U.S. Cl. 435/471; 435/320.1; 435/252.1; 530/350

(57) ABSTRACT

Plasmid comprising a DNA tag encoding a peptide tag of the sequence MX1(X 2X 3) n X 1 represents K or R; X 2 represents M, S or T; X 3 represents K or R; n represents an integer of 1 or larger; and wherein said DNA is operably-linked to a promoter sequence are provided.

### BACKGROUND OF THE INVENTION

Recombinant protein expression systems facilitate the production of proteins, polypeptides and peptides to be used as biopharmaceuticals or as targets in drug screening for a wide range of applications. Bacterial expression systems have been the preferred method of choice, largely due to the efficient and economic production in bacteria, although yeast and baculovirus provide reliable alternative expression systems.

Despite the wide use of recombinant expression systems for the production of heterologous proteins, available methods cannot be relied upon to produce any given protein in sufficient yields and having sufficient homogeneity to meet downstream requirements. Many mammalian proteins are expressed in bacteria in low yields and with a rather poor solubility. Also, they may be toxic to the bacterial cells, especially if they are partially soluble. A number of vector systems are designed to express the target recombinant protein as a fusion protein with a short or a longer N-terminal peptide tag. Examples of such tags are the histidine-, or maltose binding-tags, which are particularly useful for the subsequent purification of the recombinant proteins. There remains however a need for an efficient expression system, especially for therapeutic proteins which proteins are potentially toxic and difficult to express. Since protein yield is very much dependant on transcriptional and translational start, such a system should have an N-terminal tag conferring a high yield and a fusion protein with low solubility, since inclusion bodies are generally much better tolerated by the host. Also, the introduced tag should be readily cleavable for production of the native protein.

### SUMMARY OF THE INVENTION

The invention provides a self-replicating DNA plasmid for recombinant expression of an N-terminally tagged protein in a microbial host cell comprising a DNA tag having a nucleotide sequence encoding a peptide tag of formula [I]

MX₁(X₂X₃)ₙ [I]

wherein
X₁ represents K or R;
X₂ represents M, S or T;
X₃ represents K or R;
n represents an integer of 1 or larger;
and wherein said DNA is operably-linked to a promoter sequence.

A plasmid according to the invention may further comprise a nucleic acid sequence encoding a protein fused in-frame with said DNA tag for recombinant expression of an N-terminally tagged protein encoded by said nucleic acid fused to said DNA tag.

The invention provides a microbial host cell comprising the DNA plasmid of the invention.

In one embodiment, the invention provides a tagged protein comprising an N-terminal peptide tag fused to a protein, wherein said tag has a sequence according to formula I.

In one embodiment, the invention provides a method for recombinant expression of an N-terminally tagged protein in a microbial host cell comprising the steps of constructing a recombinant plasmid comprising inserting a DNA sequence encoding a protein in-frame and 3′ to the DNA tag of the plasmid according to the present invention, and introducing said recombinant plasmid into a host microbial cell, and inducing expression of said N-terminally tagged protein in a microbial host cell.

### DESCRIPTION OF THE DRAWINGS

FIG. 1: The efficiency and completion of tag removal to yield mature hIL-21 was determined by mass spectrometry, as shown in FIGS. 1, A and B. Panel A shows the Maldi spectrum of fractions prior to tag removal. Panel B shows the same fractions after tag removal.

FIG. 2: 2A: Maldi mass spectrum prior to DAP/Q-cyclase treatment of MKMK-IL21. Single charged molecular ion with a value of 15948 Da, corresponding to intact MKMK-IL21. 2B: Maldi mass spectrum after DAP/Q-cyclase treatment of MKMK-IL21. Single charged molecular ion with a value of 15423 Da, corresponding to IL21 with an N-terminal pyroglutamate residue. No signals corresponding to intact MKMK-IL21 was observed.

FIG. 3: 3A: Maldi mass spectrum prior to DAP/Q-cyclase treatment of MKSK-IL21. Single charged molecular ion with a value of 15911.6 Da, corresponding to intact MKSK-IL21. 3B: Maldi mass spectrum after DAP/Q-cyclase treatment of MKSK-IL21. Single charged molecular ion with a value of 15429 Da, corresponding to IL21 with an N-terminal pyroglutamate residue. No signals corresponding to intact MKSK-IL21 was observed.

FIG. 4: 4A: Maldi mass spectrum prior to DAP/Q-cyclase treatment of MKTK-IL21. Single charged molecular ion with a value of 15933.6 Da, corresponding to intact MKTK-IL21. 4B: Maldi mass spectrum after DAP/Q-cyclase treatment of MKTK-IL21. Single charged molecular ion with a value of 15430.9 Da, corresponding to IL21 with an N-terminal pyroglutamate residue. No signals corresponding to intact MKTK-IL21 was observed.

### ABBREVIATIONS

Amino acid: Alanine (A); arginine (R); asparagine (N); aspartic acid (D); cysteine (C); glycine (G); glutamine (Q); glutamic acid (E); histidine (H); isoleucine (I); leucine (L); lysine (K); methionine (M); phenylalanine (F); proline (P); serine (S); threonine (T); tryptophan (W), tyrosine (Y); valine (V).
C-terminal: carboxy (C)-terminal part of a protein, comprising one or more amino acid residues.
hIL-21: human interleukin-21
N-terminal: amino (N)-terminal part of a protein, comprising one or more amino acid residues.
SDS PAGE: sodium dodecyl (lauryl) sulfate-polyacrylamide gel

### DESCRIPTION OF THE INVENTION

The present invention provides a DNA tag, an expression-vector or -plasmid suitable for the recombinant expression of a heterologous protein, and a method for recombinant protein expression, which are compatible with the subsequent purification of the recombinant protein, and eventual processing of the recombinant protein to recover the protein in its native and active form.

Proteins expressed with an N-terminal tag according to the present invention are have a low solubility and will this preferentially be expressed into inclusion bodies, which are generally much better tolerated by the host.

In one embodiment, the present invention provides a self-replicating DNA plasmid for recombinant expression of an N-terminally tagged protein in a microbial host cell, which plasmid comprises a DNA tag having a nucleotide sequence encoding a peptide tag of formula [I]

MX₁(X₂X₃)ₙ [I]

wherein
X₁ represents K or R;
X₂ represents M, S or T;
X₃ represents K or R;
n represents an integer of 1 or larger;
and wherein said DNA tag is operably-linked to a promoter sequence.

The terms “protein”, “polypeptide” and “peptide” are used interchangeably herein and should be taken to mean a compound composed of at least five constituent amino acids connected by peptide bonds. The constituent amino acids may be from the group of the amino acids encoded by the genetic code and they may be natural amino acids which are not encoded by the genetic code, as well as synthetic amino acids. Natural amino acids which are not encoded by the genetic code are e.g. hydroxyproline, y-carboxyglutamate, ornithine, phosphoserine, D-alanine and D-glutamine. Synthetic amino acids comprise amino acids manufactured by chemical synthesis, i.e. D-isomers of the amino acids encoded by the genetic code such as D-alanine and D-leucine, Aib (a-aminoisobutyric acid), Abu (a-aminobutyric acid), Tle (tert-butylglycine), β-alanine, 3-aminomethyl benzoic acid and anthranilic acid.

As used herein, the term “DNA tag” is defined as a DNA molecule encoding an N-terminal protein tag that is added to a DNA sequence coding for a heterologous protein, and whose in frame expression in a micro-organism produces a tagged protein or fusion protein. The DNA tag of the present invention codes for an amino acid sequence having at least four amino acids and comprising an amino acid sequence as defined by formula I.

In one embodiment, X₁ represents K. In one embodiment, X₁ represents R.

In one embodiment, X₂ represents M or S. In one embodiment, X₂ represents M or T. In one embodiment, X₂ represents S or T. In one embodiment, X₂ represents M. In one embodiment, X₂ represents S. In one embodiment, X₂ represents T.

In one embodiment, X₃ represents K. In one embodiment, X₃ represents R.

In one embodiment, n is an integer of from 1 to 10. In one embodiment, n is an integer of from 1 to 9. In one embodiment, n is an integer of from 1 to 8. In one embodiment, n is an integer of from 1 to 7. In one embodiment, n is an integer of from 1 to 6. In one embodiment, n is an integer of from 1 to 5. In one embodiment, n is an integer of from 1 to 4. In one embodiment, n is an integer of from 1 to 3. In one embodiment, n is an integer of from 1 to 2. In one embodiment, n is 1. In one embodiment, n is 2. In one embodiment, n is 3.

As illustrated in the examples, the expression of a DNA sequence comprising a DNA tag of the invention, fused in-frame to the coding sequence of a protein, facilitates significantly higher levels of expression of the protein than a control sequence encoding the protein fused to an N-terminal methionine. While not wishing to be bound by theory, it is believed that recombinant protein expression in a host microbial cell, in particular an E. coli cell, is enhanced if the expressed protein accumulates in a form that is non-toxic to host cell metabolism or growth, for example in an inclusion body. Thus the selected N-terminal protein tags fused to recombinant proteins may enhance their expression by facilitating their accumulation in inclusion bodies.

Many mammalian proteins of interest are secreted in their natural host and synthesized with a signal peptide, which is cleaved off during secretion. The N-terminal of the secreted, mature protein therefore in most cases begins with an amino acid different from methionine, the natural N-terminal of all de novo synthesized proteins, including heterologous, intracellularly accumulated proteins in E. coli. To avoid uncertainties about cleavage of the N-terminal methionine, the addition of a small peptide tag as described, with known in vitro cleavage properties, is highly advantageous in obtaining the mature protein of interest.

The DNA tag provided by the present invention may be added to a DNA sequence encoding a protein for the purposes of its recombinant expression in a host microbial cell, in particular a bacterial cell. The DNA tag has application in the recombinant expression of a wide number of useful proteins in a host microbial cell, in particular for the expression of therapeutic proteins, for example human growth hormone, IL-20, IL-21, and GLP-1. The DNA tag encoding the N-terminal peptide tag is fused in-frame with the DNA sequence encoding the protein to be expressed, such that the expression product obtainable in a host cell is a tagged- or fusion-protein. If the DNA tag encodes an N-terminal peptide tag that is more that four amino acids, the peptide tag may be extended by the addition of dipeptides, whose amino acid composition is compatible with their cleavage by a diaminopeptidase, such as dipeptidyl amino peptidase I. The expressed tagged- or fusion-protein may comprise the peptide tag fused directly to the first amino acid of the mature protein to be expressed, such that cleavage of the peptide tag with the removal of dipeptides releases the expressed protein in its mature form. In the event that the peptide tag of the expressed tagged- or fusion-protein is to be removed by an aminopeptidase, it is desirable to ensure that the amino acid sequence of the mature form of the expressed protein starts with, or is preceded by, a residue that can function as a stop point beyond which the aminopeptidase can not continue. In this manner the mature form of the expressed protein is protected from N-terminal proteolytic cleavage. A suitable amino acid residue that can act as a stop point for a diaminopeptidase may be selected from Q, P, R, K. The amino acid residue Q can be used as the stop point, by virtue of its ability to form pyroglutamate in the presence of glutamate cylcotransferase. In the event that the N-terminal amino acid of the mature protein is not itself a residue that can function as a stop, it is desirable to extend the DNA tag by one codon encoding a suitable stop residue, which is then fused to the DNA sequence encoding the desired mature protein. A preferred stop residue to be added to the end of the peptide tag is Q, since this residue can be removed from the N-terminus of the expressed protein with pyroglutamyl aminopeptidase, following dipeptidyl aminopeptidase cleavage of the peptide tag.

The DNA tag of the invention when fused in-frame to the coding sequence of a protein to be recombinantly expressed, provides a tagged-protein whose peptide tag has a predominance of charged polar side chains. The presence of additional charged residues in the tagged protein may be particularly useful in subsequent purification steps that discriminate on the basis of protein mass charge.

A DNA tag according to the present invention may be fused in-frame to a DNA sequence encoding hIL-21. In one example of the invention the DNA tag according to the present invention is fused in-frame to a DNA molecule encoding hIL-21 having the nucleotide sequence of SEQ ID No. 4. Other restriction sites may be chosen, and it lies within the capabilities of a person skilled in the art to adjust the sequences accordingly.

In one aspect, the invention provides an expression-vector or -plasmid comprising a DNA tag encoding the peptide tag of the invention. The DNA tag may be inserted adjacent to, or in, a suitable cloning site of the vector or plasmid, such that the tag is located downstream and operably-linked to a promoter sequence. Preferably the DNA tag is flanked by a restriction-enzyme cleavage site that facilitates the down-stream in-frame insertion of a DNA sequence encoding the protein to be recombinantly expressed. One skilled in the art will readily recognise suitable preferred flanking sequences to facilitate downstream in-frame cloning of the coding sequence of a desired protein. A promoter sequence in the plasmid or vector of the invention, that is operably-linked to the DNA-tag of the invention, has a nucleotide sequence that is capable of directing transcription of the DNA molecule encoding the tagged protein in the selected host microbial cell. Promoter sequences, suitable for recombinant protein expression in bacteria and in particular in E. coli, are well known to one skilled in the art, but include any one of the T7, trc lac and tac promoters. A preferred vector incorporating the expression cassette comprising a promoter operably-linked to the DNA-tag of the invention is one that is self-replicating and has a selectable maker, for example ampicillin.

In one embodiment, the expression-vector or -plasmid of the invention further comprises a DNA sequence encoding a protein to be recombinantly expressed, where the DNA sequence is cloned downstream and in-frame with said DNA tag. In one example, the DNA sequence cloned in the expression plasmid is one that encodes hIL-21 that is capable of expression as a tagged protein when the expression plasmid is introduced into a suitable host cell. The DNA sequence encoding hIL-21 in the expression-vector or -plasmid of the invention may comprise the nucleotide sequence of SEQ ID No. 4.

A host cell, to be transformed with the expression-plasmid -vector of the invention, that is suitable for the expression of a tagged protein, is well-known to one skilled in the art. A preferred bacterial host stain is a derivative strain of E. coli B, for example the protease-deficient strain E. coli BL21 (DE3) habouring the T7 polymerase gene on the chromosome.

The present invention provides a tagged protein comprising an N-terminal peptide tag fused to a protein, wherein said tag comprises an amino acid sequence of formula [I]

MX₁(X₂X₃)ₙ [I]

wherein
X₁ represents K or R;
X₂ represents M, S or T;
X₃ represents K or R; and
n represents an integer of 1 or larger.

In one embodiment, X₁ represents K. In one embodiment, X₁ represents R.

In one embodiment, X₂ represents M or S. In one embodiment, X₂ represents M or T. In one embodiment, X₂ represents S or T. In one embodiment, X₂ represents M. In one embodiment, X₂ represents S. In one embodiment, X₂ represents T.

In one embodiment, X₃ represents K. In one embodiment, X₃ represents R.

In one embodiment, n is an integer of from 1 to 10. In one embodiment, n is an integer of from 1 to 9. In one embodiment, n is an integer of from 1 to 8. In one embodiment, n is an integer of from 1 to 7. In one embodiment, n is an integer of from 1 to 6. In one embodiment, n is an integer of from 1 to 5. In one embodiment, n is an integer of from 1 to 4. In one embodiment, n is an integer of from 1 to 3. In one embodiment, n is an integer of from 1 to 2. In one embodiment, n is 1. In one embodiment, n is 2. In one embodiment, n is 3.

In one embodiment, said protein comprises an amino acid sequence of SEQ ID No.-2.

In one embodiment, said peptide tag is not MKMK, MKTK or MKSK.

The tagged protein according to the present invention can be obtained by recombinant expression of the expression-plasmid or -vector of the present invention. The tagged protein may be subjected to purification steps, and/or one or more proteolytic processing steps described herein for the removal of the peptide tag from the tagged protein in order to provide a mature protein having one or more applications.

The invention further provides a method for recombinant expression in a host microbial cell of a tagged protein encoded by a DNA tag of the invention fused in-frame to a coding sequence, whereby the fused DNA sequence encodes said tagged protein, in order to improve the yield of the expressed target protein. Accordingly, the method includes the steps of constructing an expression-plasmid or -vector coding for a fusion protein which comprises an N-terminal peptide tag fused to a protein, whereby the coding sequence is terminated by a stop codon. Expression of the tagged protein is directed by a promoter operably-linked to the coding sequence of the tagged protein, whereby the promoter is one that is recognised by the expression system of the host cell. According to one embodiment of the invention, the construction of an expression-vector for the expression of hIL-21 is described in example 1.

The expression-vector or -plasmid of the invention is transfected into a host microbial cell, preferably the bacterium E. coli and host cells transformed by the vector are identified, isolated and cultivated under conditions compatible with multiplication of the host cell and the expression of the tagged protein.

Expression of the tagged protein of the invention in a host microbial cell is preferably inducible. For example, where the host cell is an E. coli strain, and expression is regulated by the lac operator, expression may be induced by addition of about 0.5-1 mM isopropyl β-D-thiogalactopyranoside (IPTG) that de-represses the lac promoter. After a suitable induction by IPTG, for example for 3-4 hours, the host cells may be lysed, for example by sonication or freese-thaw procedures, and the cell lysate separated into soluble and insoluble fractions by centrifugation. The tagged protein, depending on its solubility, may be located in the soluble fraction, or more preferably in inclusion bodies that fractionate with the cell pellet.

When the tagged protein is located in inclusion bodies, a solubilisation and refolding step may be required prior to its further purification, employing conditions optimized for the tagged protein according to protocols well known in the art. A wide variety of protein separation and purification protocols may be employed to achieve the required degree of purification. Methods for determining the purity of the purified tagged protein of the invention and the subsequently derived mature protein are well known in the art, and are illustrated in Example 2.

Removal of the peptide tag from the tagged protein of the invention may employ di-peptidyl aminopeptidase, which may be combined with glutamine cyclotransferase if the stop residue is Q. Removal of the tag may be performed either before or after purification of the recombinantly expressed protein of the invention.

The following is a list of embodiments of the present invention, which should not be construed as limiting.

### Embodiment 1

A self-replicating DNA plasmid for recombinant expression of an N-terminally tagged protein in a microbial host cell, which plasmid comprises a DNA tag having a nucleotide sequence encoding a peptide tag of formula [I]

MX₁(X₂X₃)ₙ [I]

wherein
X₁ represents K or R;
X₂ represents M, S or T;
X₃ represents K or R;
n represents an integer of 1 or larger;
and wherein said DNA tag is operably-linked to a promoter sequence.

### Embodiment 2

A DNA plasmid according to embodiment 1, wherein X₁ represents K.

### Embodiment 3

A DNA plasmid according to embodiment 1, wherein X₁ represents R.

### Embodiment 4

A DNA plasmid according to any of embodiments 1 to 3, wherein X₂ represents M or S.

### Embodiment 5

A DNA plasmid according to any of embodiments 1 to 3, wherein X₂ represents M or T.

### Embodiment 6

A DNA plasmid according to any of embodiments 1 to 3, wherein X₂ represents S or T.

### Embodiment 7

A DNA plasmid according to any of embodiments 1 to 3, wherein X₂ represents M.

### Embodiment 8

A DNA plasmid according to any of embodiments 1 to 3, wherein X₂ represents S.

### Embodiment 9

A DNA plasmid according to any of embodiments 1 to 3, wherein X₂ represents T.

### Embodiment 10

A DNA plasmid according to any of embodiments 1 to 9, wherein X₃ represents K.

### Embodiment 11

A DNA plasmid according to any of embodiments 1 to 9, wherein X₃ represents R.

### Embodiment 12

A DNA plasmid according to any of embodiments 1 to 11, wherein n is 1.

### Embodiment 13

A DNA plasmid according to any of embodiments 1 to 11, wherein n is 2.

### Embodiment 14

A DNA plasmid according to any of embodiments 1 to 11, wherein n is 3.

### Embodiment 15

A plasmid according to any of embodiments 1 to 14, further comprising a nucleic acid sequence encoding a protein fused in-frame with said DNA tag for recombinant expression of an N-terminally tagged protein encoded by said nucleic acid sequence fused to said DNA tag.

### Embodiment 16

A plasmid according to embodiment 15, wherein the expression of the protein by use of said plasmid is increased as compared to the expression of the protein without said peptide tag.

### Embodiment 17

A plasmid according to embodiment 15 or 16, wherein the solubility of the protein expressed by use of said plasmid is decreased as compared to the solubility of the protein expressed without said peptide tag.

### Embodiment 18

A plasmid according to any of embodiments 15 to 17, wherein said protein comprises the amino acid sequence of SEQ ID No. 2.

### Embodiment 19

A plasmid according to any of embodiments 15 to 18, wherein said nucleic acid sequence encoding a protein consists of the nucleotide sequence of SEQ ID No. 1.

### Embodiment 20

A DNA plasmid according to any of embodiments 1 to 19, with the provisio that the peptide tag encoded by the DNA tag is not MKMK, MKTK or MKSK.

### Embodiment 21

A microbial host cell comprising a plasmid according to any one of embodiments 1 to 20.

### Embodiment 22

A microbial host cell according to embodiment 21, wherein said cell is E. coli.

### Embodiment 23

A tagged protein comprising an N-terminal peptide tag fused to a protein, wherein said tag comprises an amino acid sequence of formula [I]

MX₁(X₂X₃)ₙ [I]

wherein
X₁ represents K or R;
X₂ represents M, S or T;
X₃ represents K or R; and
n represents an integer of 1 or larger.

### Embodiment 24

A tagged protein according to embodiment 23, wherein X₁ represents K.

### Embodiment 25

A tagged protein according to embodiment 23, wherein X₁ represents R.

### Embodiment 26

A tagged protein according to any of embodiments 23 to 25, wherein X₂ represents M or S.

### Embodiment 27

A tagged protein according to any of embodiments 23 to 25, wherein X₂ represents M or T.

### Embodiment 28

A tagged protein according to any of embodiments 23 to 25, wherein X₂ represents S or T.

### Embodiment 29

A tagged protein according to any of embodiments 23 to 25, wherein X₂ represents M.

### Embodiment 30

A tagged protein according to any of embodiments 23 to 25, wherein X₂ represents S.

### Embodiment 31

A tagged protein according to any of embodiments 23 to 25, wherein X₂ represents T.

### Embodiment 32

A tagged protein according to any of embodiments 23 to 31, wherein X₃ represents K.

### Embodiment 33

A tagged protein according to any of embodiments 23 to 31, wherein X₃ represents R.

### Embodiment 34

A tagged protein according to any of embodiments 23 to 33, wherein n is 1.

### Embodiment 35

A tagged protein according to any of embodiments 23 to 33, wherein n is 2.

### Embodiment 36

A tagged protein according to any of embodiments 23 to 33, wherein n is 3.

### Embodiment 37

A tagged protein according to any of embodiments 23 to 36, wherein said protein comprises an amino acid sequence of SEQ ID No. 2.

### Embodiment 38

A tagged protein according to any of embodiments 23 to 37, with the provisio that the peptide tag is not MKMK, MKTK or MKSK.

### Embodiment 39

A method for recombinant expression of an N-terminally tagged protein in a microbial host cell comprising the steps of:
(a) constructing a recombinant plasmid comprising inserting a DNA sequence encoding a protein in-frame and 3′ to the DNA tag of a plasmid according to any one of embodiments 1 to 14, and
(b) introducing said recombinant plasmid into a host microbial cell, and
(c) inducing expression of said N-terminally tagged protein in a microbial host cell.

### Embodiment 40

A method for increasing the recombinant expression of a protein in a microbial host cell, which method comprises
(a) constructing a recombinant plasmid comprising inserting a DNA sequence encoding the protein in-frame and 3′ to the DNA tag of a plasmid according to any one of embodiments 1 to 14, and
(b) introducing said recombinant plasmid into a host microbial cell, and
(c) inducing expression of said N-terminally tagged protein in a microbial host cell.

### Embodiment 41

A method for decreasing the solubility of a recombinantly expressed protein in a microbial host cell, which method comprises
(a) constructing a recombinant plasmid comprising inserting a DNA sequence encoding the protein in-frame and 3′ to the DNA tag of a plasmid according to any one of embodiments 1 to 14, and
(b) introducing said recombinant plasmid into a host microbial cell, and
(c) inducing expression of said N-terminally tagged protein in a microbial host cell.

### Embodiment 42

A method according to any of embodiments 39 to 41, wherein said protein comprises the amino acid sequence of SEQ ID No. 2.

### Embodiment 43

A method according to any of embodiments 39 to 42, wherein the DNA sequence encoding the protein consists of the nucleotide sequence of SEQ ID No. 1.

### Embodiment 44

A method according to any of embodiments 39 to 43, with the provisio that the peptide tag encoded by the DNA tag of the plasmide not MKMK, MKTK or MKSK.

All references, including publications, patent applications, and patents, cited herein are hereby incorporated by reference in their entirety and to the same extent as if each reference were individually and specifically indicated to be incorporated by reference and were set forth in its entirety herein (to the maximum extent permitted by law), regardless of any separately provided incorporation of particular documents made elsewhere herein.

The use of the terms “a” and “an” and “the” and similar referents in the context of describing the invention are to be construed to cover both the singular and the plural, unless otherwise indicated herein or clearly contradicted by context. For example, the phrase “the compound” is to be understood as referring to various “compounds” of the invention or particular described aspect, unless otherwise indicated.

Unless otherwise indicated, all exact values provided herein are representative of corresponding approximate values (e.g., all exact exemplary values provided with respect to a particular factor or measurement can be considered to also provide a corresponding approximate measurement, modified by “about,” where appropriate).

The description herein of any aspect or aspect of the invention using terms such as “comprising”, “having,” “including,” or “containing” with reference to an element or elements is intended to provide support for a similar aspect or aspect of the invention that “consists of”, “consists essentially of”, or “substantially comprises” that particular element or elements, unless otherwise stated or clearly contradicted by context (e.g., a composition described herein as comprising a particular element should be understood as also describing a composition consisting of that element, unless otherwise stated or clearly contradicted by context).

In summary, the present invention provides an expression-vector or -plasmid comprising a DNA tag encoding a peptide tag, that is operably-linked to a promoter capable of directing expression in a host microbial cell of said DNA tag and any protein coding sequence fused in-frame with said DNA tag. The particular advantage of employing the expression-vector or -plasmid of the invention for recombinant protein expression of a protein coding sequence fused in-frame with said DNA tag is that the expression levels in a host cell are significantly enhanced. Thus, when a protein is recombinantly expressed in a microbial host cell, such as e.g. E. coli, with the peptide tag of the invention fused at the N-terminus, the presence of this tag in most cases enhances expression, due to decreased solubility of the protein and reduced toxicity to the host cell, and it further fulfils a number of additional important criteria required for efficient recombinant protein expression. In particular it allows the protein to be obtained in its mature form after proper cleavage of the tag. Moreover, the alteration of the overall protein charge brought about by the charged tag facilitates the purification of the protein.

### EXAMPLES

### Example 1

### Expression of Tagged Human Interleukin-21

For comparison of various small N-terminal tags, with respect to expression and down-stream processing, the human interleukin hIL-21 was chosen as the target protein. The nucleic acid molecule encoding the protein hIL-21 is SEQ ID No. 1 (Met hIL-21 Nde1-BamH1 nucleotide sequence), where the 5′ end and 3′ end of the molecule has respectively restriction enzyme sites for Nde1-BamH1.

The Met hIL-21 Nde1-BamH1 nucleotide sequence encodes the hIL-21 protein sequence shown in SEQ ID No. 2. When expressed in E. coli, this protein has an additional methionine in the N-terminal. This version of hIL21 is called Met-hIL21.

A series of constructs were made according to the following scheme:

A 410 base pair DNA molecule, encoding the mature form of hIL-21, corresponding to amino acid residues 1-133 of Met-hIL-21, with 5′ and 3′ end Sty1-BamH1 sites is shown in SEQ ID No. 3 (hIL-21 Sty1-BamH1 nucleotide sequence). The hIL-21 Sty1-BamH1 nucleotide sequence, starting from nucleotide 2, comprises the nucleotide sequence as shown in SEQ ID No. 4, which codes for the mature hIL-21 protein sequence having amino acid sequence of SEQ ID No. 2.

The hIL-21 Sty1-BamH1 molecule was ligated to an Nde1-BamH1 digested T7 expression vector, pET-11c of 5.6 kb, together with any one of a series of linkers, each flanked by a 5′ Nde1 site and a 3′ Sty1 compatible site, that are listed in Table 1.

TABLE 1

| | | | |
|---|---|---|---|
| | Amino acid | | |
| Name of | sequence | Expression | |
| construct | of tag | level | DNA sequence of tag* |

| | | | | |
|---|---|---|---|---|
| Met hIL-21 | (M) | 1-2 | No tag | |
| DAP 21 | MKMK | 4 | 5′T ATG AAA ATG AAA 3′ | [SEQ ID No: 5] |
| | (SEQ ID No. 6) | | AC TTT TAC TTT GTT C | |
| DAP 23 | MKSK | 6 | 5′T ATG AAA AGC AAA 3′ | [SEQ ID No: 7] |
| | (SEQ ID No. 8) | | AC TTT TCG TTT GTT C | |
| DAP 24 | MKTK | 4 | 5′T ATG AAA ACC AAA 3′ | [SEQ ID No: 9] |
| | (SEQ ID No. 10) | | AC TTT TGG TTT GTT C | |

The T7 expression vector, pET-11c, comprising a linker containing a DNA tag, ligated in-frame to the DNA molecule, hIL-21 Sty1-BamH1 was transformed into the host cell E. coli B BL21 (DE3).

Host cell strains, transformed with each of the T7 expression vectors, were grown at 37° C. in LB medium, supplemented with ampicillin 0.2 mg/l, and recombinant protein expression from the T7 expression vector was induced with 0.5 mM IPTG for 3-4 hours. The host cells were then harvested by centrifugation, lysed and then the sample was centrifuged to provide a soluble fraction and a pelleted inclusion body fraction. The total cell extract, the inclusion body and soluble cell fraction from each host cell sample was then separated by SDS PAGE, and the gels were stained with Comassie blue to determine the relative level of tagged hIL-21 protein expression, as compared with the untagged protein, Met hIL-21.

The expression level of the various tagged versions of hIL-21 is dependant on the amino acid sequence of the tag, as shown in Table 1, but it is also in some cases dependant on the nucleotide sequence, i.e. secondary structure in the mRNA. It is within the skills of a person skilled in the art to make adjustments to the codons to avoid secondary problems if encountered. Table 1 illustrates two points: The expression levels are generally increased by the addition of the specific peptide tags, and the solubility of hIL-21 is decreased thereby protecting the E. coli host cell from the poisonous effects of hIL-21. Also, the decrease in solubility favours the partitioning of hIL-21 into inclusion bodies and thereby facilitates its subsequent purification.

### Example 2

### Recombinantly Expressed Tagged Human Interleukin-21 is Processed to its Mature and Active Form

MKHK-hIL-21, expressed using construct DAP17, was refolded from inclusion bodies as disclosed in WO 04/55168 and subsequently purified to approximately 90-95% purity employing Sepharose SP column chromatography. A single major polypeptide band corresponding to MKHK-hIL-21 was detected by SDS-PAGE analysis of fractions obtained from the Sepharose SP column and pools of fractions, shown in lanes 4-10, were subsequently subjected to dipeptidyl aminopeptidase (DAPase) and glutamine cyclotransferase (Q cyclase) treatment in order to perform a controlled removal of the N-terminal peptide tag of four amino acids. The conditions for peptide tag cleavage were: an aqueous solution of 27.5 μM MKHK-IL21, 67.5 mU DAPase, 5.5 U Q cyclase, 25 mM Tris, 0.15 M NaCl, pH 7.0, incubated for 90 minutes at ambient temperature (20-25° C.), employing enzymes supplied by Qiagen.com.

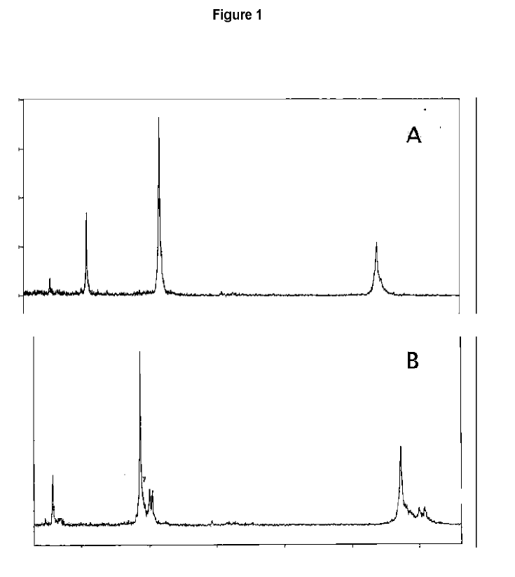

The efficiency and completion of tag removal to yield mature hIL-21 was determined by mass spectrometry, as shown in FIGS. 1, A and B.

Panel A shows the Maldi spectrum of fractions prior to tag removal

Panel B shows the same fractions after tag removal.

Native hIL21 have a molecular weight of 15433 Da, while the MKHK-IL21 has a molecular weight of 15975 Da. As observed in panel B, cleavage and tag removal was approximately 90% complete.

### Example 3

### Recombinantly Expressed MKMK-Tagged Human Interleukin-21 is Processed to its Mature and Active Form

MKMK-hIL-21, expressed using construct DAP21, was refolded from inclusion bodies as disclosed in WO200455168 and subsequently purified to approximately 90-95% purity employing TosoHaas sp 550c column chromatography. A single major polypeptide band corresponding to MKMK-hIL-21 was detected by SDS-PAGE analysis of fractions obtained from the TosoHaas sp 550c column and pools of fractions were subsequently subjected to dipeptidyl aminopeptidase (DAPase) and glutamine cyclotransferase (Q cyclase) treatment in order to perform a controlled removal of the N-terminal peptide tag of four amino acids. The conditions for peptide tag cleavage were: an aqueous solution of 2 mg/ml MKMK-IL21 and a molar ratio of MKMK-IL21:DAPase:Q cyclase of 800:1:32 in 25 mM Tris, 0.15 M NaCl, pH 7.0, incubated for 30 minutes at ambient temperature (20-25° C.), employing enzymes supplied by Qiagen.com.

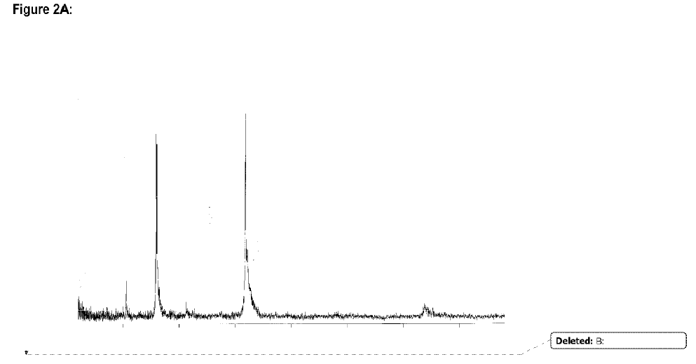

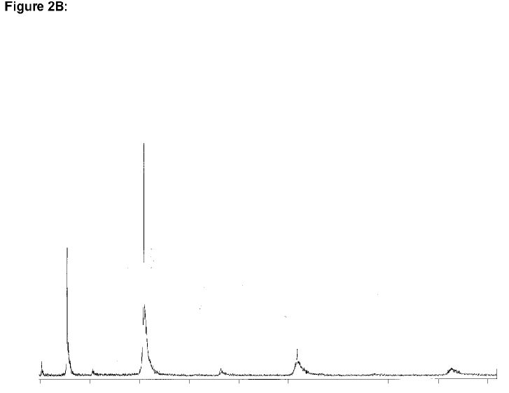

The efficiency and completion of tag removal to yield mature hIL-21 was determined by mass spectrometry, as shown in FIGS. 2, A and B. Panel A shows the Maldi spectrum of fractions prior to tag removal. Panel B shows the same fractions after tag removal.

Native hIL21 with an N-terminal pyroglutamate have a molecular weight of 15442 Da, while the MKMK-IL21 has a molecular weight of 15978 Da. As observed in panel B, cleavage and tag removal was complete.

### Example 4

### Recombinantly Expressed MKSK-Tagged Human Interleukin-21 is Processed to its Mature and Active Form

MKSK-hIL-21, expressed using construct DAP23, was refolded from inclusion bodies as disclosed in WO200455168 and subsequently purified to approximately 90-95% purity employing TosoHaas sp 550c column chromatography. A single major polypeptide band corresponding to MKSK-hIL-21 was detected by SDS-PAGE analysis of fractions obtained from the TosoHaas sp 550c column and pools of fractions were subsequently subjected to dipeptidyl aminopeptidase (DAPase) and glutamine cyclotransferase (Q cyclase) treatment in order to perform a controlled removal of the N-terminal peptide tag of four amino acids. The conditions for peptide tag cleavage were: an aqueous solution of 2 mg/ml MKSK-IL21 and a molar ratio of MKSK-IL21:DAPase:Q cyclase of 800:1:32 in 25 mM Tris, 0.15 M NaCl, pH 7.0, incubated for 30 minutes at ambient temperature (20-25° C.), employing enzymes supplied by Qiagen.com.

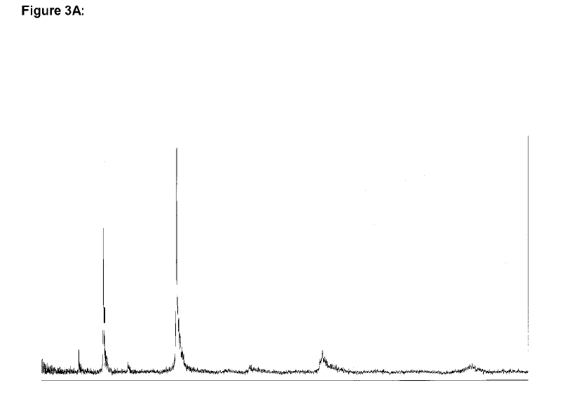

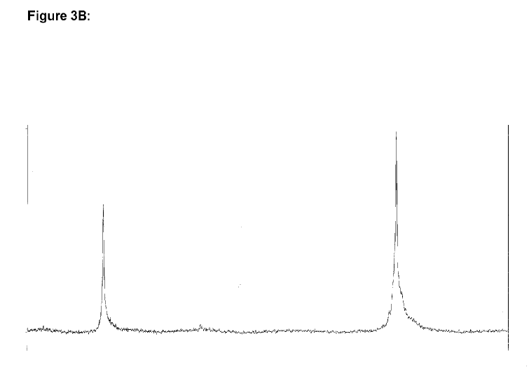

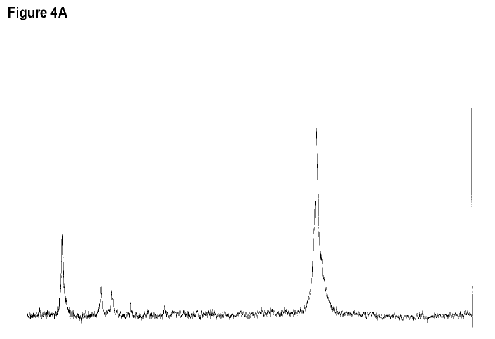

The efficiency and completion of tag removal to yield mature hIL-21 was determined by mass spectrometry, as shown in FIGS. 3, A and B. Panel A shows the Maldi spectrum of fractions prior to tag removal. Panel B shows the same fractions after tag removal.

Native hIL21 with an N-terminal pyroglutamate have a molecular weight of 15442 Da, while the MKSK-IL21 has a molecular weight of 15934 Da. As observed in panel B, cleavage and tag removal was complete.

### Example 5

### Recombinantly Expressed MKTK-Tagged Human Interleukin-21 is Processed to its Mature and Active Form

MKTK-hIL-21, expressed using construct DAP24, was refolded from inclusion bodies as disclosed in WO 04/55168 and subsequently purified to approximately 90-95% purity employing TosoHaas sp 550c column chromatography. A single major polypeptide band corresponding to MKTK-hIL-21 was detected by SDS-PAGE analysis of fractions obtained from the TosoHaas sp 550c column and pools of fractions were subsequently subjected to dipeptidyl aminopeptidase (DAPase) and glutamine cyclotransferase (Q cyclase) treatment in order to perform a controlled removal of the N-terminal peptide tag of four amino acids. The conditions for peptide tag cleavage were: an aqueous solution of 2 mg/ml MKTK-IL21 and a molar ratio of MKTK-IL21:DAPase:Q cyclase of 800:1:32 in 25 mM Tris, 0.15 M NaCl, pH 7.0, incubated for 30 minutes at ambient temperature (20-25° C.), employing enzymes supplied by Qiagen.com.

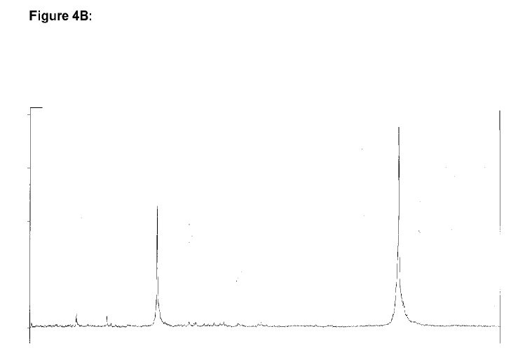

The efficiency and completion of tag removal to yield mature hIL-21 was determined by mass spectrometry, as shown in FIGS. 4, A and B. Panel A shows the Maldi spectrum of fractions prior to tag removal. Panel B shows the same fractions after tag removal.

Native hIL21 with an N-terminal pyroglutamate have a molecular weight of 15442 Da, while the MKTK-IL21 has a molecular weight of 15948 Da. As observed in panel B, cleavage and tag removal was complete.

### Pharmacological Methods

### Assay (I) BAF-3 Assay to Determine IL-21 Activity

The BAF-3 cells (a murine pro-B lymphoid cell line derived from the bone marrow) was originally IL-3 dependent for growth and survival. Il-3 activates JAK-2 and STAT which are the same mediators IL-21 is activating upon stimulation. After transfection of the human IL-21 receptor the cell line was turned into a IL-21-dependent cell line. This clone can be used to evaluate the effect of IL-21 samples on the survival of the BAF-3 cells.

The BAF-3 cells are grown in starvation medium (culture medium without IL-21) for 24 hours at 37° C., 5% CO₂.

The cells are washed and re-suspended in starvation medium and seeded on plates. 10 μl of IL-21 compound, human IL-21 in different concentrations as control is added to the cells, and the plates are incubated for 68 hours at 37° C., 5% CO₂.

AlamarBlue® is added to each well and the cells are then incubated for another 4 hours. The AlamarBlue® is a redox indicator, and is reduced by reactions innate to cellular metabolism and, therefore, provides an indirect measure of viable cell number.

Finally, the metabolic activity of the cells is measured in a fluorescence plate reader. The absorbance in the samples is expressed in % of cells not stimulated with growth hormone compound or control and from the concentration-response curves the activity (amount of a compound that stimulates the cells with 50%) can be calculated.

Biological activity of the constructs of the invention as tested in this assay using the IL-21 receptor shows that the potency of the cleaved native IL-21 from all constructs were equipotent to Met-IL21 produced by the methods described in WO200455168.

1. A self-replicating DNA plasmid for recombinant expression of an N-terminally tagged protein in a microbial host cell, which plasmid comprises a DNA tag having a nucleotide sequence encoding a peptide tag of formula [I]
MX₁(X₂X₃)ₙ [I]
wherein
X₁ represents K or R;
X₂ represents M, S or T;
X₃ represents K or R;
n represents an integer of 1 or larger;
and wherein said DNA tag is operably-linked to a promoter sequence.

2. A DNA plasmid according to claim 1, wherein n is 1, 2 or 3.

3. A plasmid according to claim 1 or claim 2, further comprising a nucleic acid sequence encoding a protein fused in-frame with said DNA tag for recombinant expression of an N-terminally tagged protein encoded by said nucleic acid sequence fused to said DNA tag.

4. A plasmid according to claim 3, wherein said protein comprises the amino acid sequence of SEQ ID No. 2.

5. A plasmid according to claim 3, wherein said nucleic acid sequence encoding a protein consists of the nucleotide sequence of SEQ ID No. 1.

6. A DNA plasmid according to claim 1 with the proviso provisio that the peptide tag encoded by the DNA tag is not MKMK, MKTK or MKSK.

7. A microbial host cell comprising a plasmid according to claim 1.

8. A tagged protein comprising an N-terminal peptide tag fused to a protein, wherein said tag comprises an amino acid sequence of formula [I]
MX₁(X₂X₃)ₙ [I]
wherein
X₁ represents K or R;
X₂ represents M, S or T;
X₃ represents K or R; and
n represents an integer of 1 or larger.

9. A tagged protein according to claim 8, wherein n is 1, 2 or 3.

10. A tagged protein according to claim 8, wherein said protein comprises an amino acid sequence of SEQ ID No. 2.

11. A tagged protein according to claim 8, with the proviso that the peptide tag is not MKMK, MKTK or MKSK.

12. A method for recombinant expression of an N-terminally tagged protein in a microbial host cell comprising the steps of:
(a) constructing a recombinant plasmid comprising inserting a DNA sequence encoding a protein in-frame and 3′ to the DNA tag of a plasmid according to claim 1, and
(b) introducing said recombinant plasmid into a host microbial cell, and
(c) inducing expression of said N-terminally tagged protein in a microbial host cell.

13. A method for increasing the recombinant expression of a protein in a microbial host cell, which method comprises
(a) constructing a recombinant plasmid comprising inserting a DNA sequence encoding the protein in-frame and 3′ to the DNA tag of a plasmid according to claim 1, and
(b) introducing said recombinant plasmid into a host microbial cell, and
(c) inducing expression of said N-terminally tagged protein in a microbial host cell.

14. A method for decreasing the solubility of a recombinantly expressed protein in a microbial host cell, which method comprises
(a) constructing a recombinant plasmid comprising inserting a DNA sequence encoding the protein in-frame and 3′ to the DNA tag of a plasmid according to claim 1, and
(b) introducing said recombinant plasmid into a host microbial cell, and
(c) inducing expression of said N-terminally tagged protein in a microbial host cell.

15. A method according to claim 12, wherein said protein comprises the amino acid sequence of SEQ ID No. 2.

16. A method according to claim 12, wherein the DNA sequence encoding the protein consists of the nucleotide sequence of SEQ ID No. 1.

17. A method according to claim 12, with the proviso that the peptide tag encoded by the DNA tag of the plasmid is not MKMK, MKTK or MKSK.

18. A method according to claim 13, wherein said protein comprises the amino acid sequence of SEQ ID No. 2.

19. A method according to claim 14, wherein said protein comprises the amino acid sequence of SEQ ID No. 2.

20. A method for recombinant expression of an N-terminally tagged protein in a microbial host cell comprising the steps of:
(a) constructing a recombinant plasmid comprising inserting a DNA sequence encoding a protein in-frame and 3′ to the DNA tag of a plasmid according to claim 2, and
(b) introducing said recombinant plasmid into a host microbial cell, and
(c) inducing expression of said N-terminally tagged protein in a microbial host cell.
